(19) United States
(12) Patent Application Publication
(10) Pub. No.: US 2022/0258155 A1
(43) Pub. Date: Aug. 18, 2022

(54) LATERAL FLOW ASSAY WITH SAMPLE ADEQUACY LINE

(71) Applicant: Becton, Dickinson and Company, Franklin Lakes, NJ (US)

(72) Inventors: Huimiao Ren, San Diego, CA (US); Brian Dwyer, San Diego, CA (US); Richard R. Anderson, San Diego, CA (US)

(21) Appl. No.: 17/650,730

(22) Filed: Feb. 11, 2022

Related U.S. Application Data
(60) Provisional application No. 63/149,321, filed on Feb. 14, 2021.

(51) Int. Cl. B01L 3/00; G01N 33/558

(52) U.S. Cl. CPC B01L 3/5023; G01N 33/558; B01L 2400/0478; B01L 2200/0605; B01L 2300/0825; B01L 3/5029

(57) ABSTRACT

Immunoassay devices, systems, and methods described herein measure the presence of analytes of interest in a sample, as well as the presence of sample adequacy markers in a sample. The sample adequacy markers allow for the assessment of the quality, quantity, and composition of the sample. In one aspect, the sample adequacy marker in the sample includes human albumin. In another aspect, the sample adequacy marker in the sample includes lactotransferrin. The immunoassay devices, systems, and methods are able to distinguish between samples that do not contain an analyte of interest, and samples that are of insufficient adequacy to obtain an accurate result. The immunoassay devices, systems, and methods described herein can be implemented as part of a clinical, laboratory, or at-home testing system.

### CROSS-REFERENCE TO RELATED APPLICATIONS

This application claims the benefit of U.S. Provisional Application No. 63/149,321, filed Feb. 14, 2021, which is hereby incorporated by reference in its entirety.

### TECHNICAL FIELD

The present disclosure relates in general to immunoassay devices, test systems, and methods. More particularly, the present disclosure relates to an immunoassay test strip that includes a reagent test line used to indicate the adequacy of a sample, such that a negative test result due to the absence of an analyte of interest in a sample is distinguishable from a negative test result due to poor sample quality, quantity, or composition.

### BACKGROUND

Immunoassay systems described herein provide reliable, inexpensive, portable, rapid, and simple diagnostic tests. Immunoassays can quickly and accurately detect the presence or absence of, and in some cases quantify, an analyte of interest in a sample. Advantageously, most immunoassays are minimally invasive and used as point-of-care testing systems. Immunoassays have been developed to detect a wide variety of medical or environmental analytes. However, immunoassays suffer from many disadvantages, including false negatives, inaccurately low results, and lack of resolution when the analyte of interest is present in the sample at high concentrations.

### SUMMARY

Disclosed herein is an immunoassay test strip comprising a flow path configured to receive a sample, a sample receiving zone coupled to the flow path, an at least one sample adequacy line, and an at least one analyte capture line. Embodiments of the present disclosure are described with reference to an immunoassay test strip, but it will be understood that use of a sample adequacy marker to indicate the adequacy of a sample can also be implemented according to the present disclosure in immunoassays that do not use a test strip format, including, but not limited to, an assay performed using a plate-based or well-plate assay, a Luminex assay, an ELISA assay, a bead-based assay, and a microarray assay. In some embodiments, the sample comprises an at least one sample adequacy marker, the at least one sample adequacy marker in the sample comprises human albumin, and the at least one sample adequacy line includes anti-HSA antibody. In some embodiments, the sample comprises an at least one sample adequacy marker, the at least one sample adequacy marker in the sample comprises lactotransferrin, and the at least one sample adequacy line includes anti-lactotransferrin antibody. In some embodiments, the presence and abundancy of the at least one sample adequacy marker in the sample is dependent on the quantity, quality and/or composition of the sample. In some embodiments, the sample further comprises an at least one analyte of interest. In some embodiments, the presence and abundancy of the at least one analyte of interest in the sample is dependent on the disease state of the donor. In some embodiments, sample is selected from a group consisting of a nasal fluid, mucus, blood, plasma, urine, sweat, or saliva sample collected from a donor. In some embodiments, the at least one analyte of interest in the sample comprises a marker of infectious disease. In some embodiments, the at least one analyte of interest in the sample comprises a viral marker. In some embodiments, the at least one analyte of interest in the sample comprises a marker for SARS-CoV-2. In some embodiments, the at least one analyte of interest in the sample comprises a marker for influenza. In some embodiments, the at least one analyte of interest in the sample comprises a marker for influenza A virus. In some embodiments, the at least one analyte of interest in the sample comprises a marker for influenza B virus. In some embodiments, the at least one analyte of interest in the sample comprises a marker for influenza and a marker for SARS-CoV-2. In some embodiments, the at least one analyte of interest in the sample comprises a bacterial marker. In some embodiments, the donor of the sample is mammalian. In some embodiments, the donor of the sample is human. In some embodiments, the test strip is used as part of a direct immunoassay. In some embodiments, the test strip is used as part of an indirect immunoassay. In some embodiments, the test strip is used as part of a sandwich immunoassay. In some embodiments, the test strip is used as part of a competitive immunoassay. In some embodiments, the test strip is used as part of a lateral flow assay. In some embodiments, the test strip is used in a BD Veritor™ System. In some embodiments, the test strip is used in a BD Veritor™ At-Home COVID-19 Test. In some embodiments, the test strip further comprises a negative control line. In some embodiments, the test strip further comprises a positive control line. In some embodiments, the positive control line is located on the test strip between the at least one analyte capture line and the at least one sample adequacy line, between the at least one analyte capture line and the negative control line, or between the at least one sample adequacy line and the negative control line. In some embodiments, wherein the positive control line validates the integrity of the immunoassay, proper reagent function, and assures correct testing procedures. In some embodiments, the negative control line validates the integrity of the immunoassay, proper reagent function, and assures correct testing procedures. In some embodiments, the sample is unlabeled upon entry into the flow path. In some embodiments, the sample is labeled upon entry into the flow path.

Some aspects of the disclosure relate to a method for assessing the adequacy of a sample using an immunoassay test strip, the immunoassay test strip comprising a flow path configured to receive a sample, a sample receiving zone coupled to the flow path, an at least one sample adequacy line, and an at least one analyte capture line, the method comprising applying the sample to the immunoassay test strip, flowing the sample in the flow path to the sample receiving zone, monitoring for the presence of an at least one analyte of interest in the sample through an immunoassay conducted in the at least one analyte capture line, and monitoring for the presence of an at least one sample adequacy marker in the sample through an immunoassay conducted in the at least one sample adequacy line; wherein the detection of the at least one sample adequacy marker is used as an indication of the sample quantity, quality and/or composition. Embodiments of the present disclosure are described with reference to a method for assessing the adequacy of a sample using an immunoassay test strip, but it will be understood that use of a sample adequacy marker to indicate the adequacy of a sample can also be implemented according to the present disclosure in immunoassays that do not use a test strip format, including, but not limited to, an assay performed using a plate-based or well-plate assay, a Luminex assay, an ELISA assay, a bead-based assay, and a microarray assay. In some embodiments, the sample comprises an at least one sample adequacy marker, the at least one sample adequacy marker in the sample comprises human albumin, and the at least one sample adequacy line includes anti-HSA antibody. In some embodiments, the sample comprises an at least one sample adequacy marker, the at least one sample adequacy marker in the sample comprises lactotransferrin, and the at least one sample adequacy line includes anti-lactotransferrin antibody. In some embodiments, the sample further comprises an at least one analyte of interest. In some embodiments, the presence and abundancy of the at least one analyte of interest in the sample is dependent on the disease state of the donor. In some embodiments, the sample is selected from a group consisting of a nasal fluid, mucus, blood, plasma, urine, sweat, or saliva sample collected from a donor. In some embodiments, the at least one analyte of interest in the sample comprises a marker of infectious disease. In some embodiments, the at least one analyte of interest in the sample comprises a viral marker. In some embodiments, the at least one analyte of interest in the sample comprises a marker for SARS-CoV-2. In some embodiments, the at least one analyte of interest in the sample comprises a marker for influenza. In some embodiments, the at least one analyte of interest in the sample comprises a marker for influenza A virus. In some embodiments, the at least one analyte of interest in the sample comprises a marker for influenza B virus. In some embodiments, the at least one analyte of interest in the sample comprises a marker for influenza and a marker for SARS-CoV-2. In some embodiments, the at least one analyte of interest in the sample comprises a bacterial marker. In some embodiments, the donor of the sample is mammalian. In some embodiments, the donor of the sample is human. In some embodiments, the immunoassay used to monitor for the presence of the at least one analyte of interest is a direct immunoassay. In some embodiments, the immunoassay used to monitor for the presence of the at least one sample adequacy marker is a direct immunoassay. In some embodiments, the immunoassay used to monitor for the presence of the at least one analyte of interest is an indirect immunoassay. In some embodiments, the immunoassay used to monitor for the presence of the at least one sample adequacy marker is an indirect immunoassay. In some embodiments, the immunoassay used to monitor for the presence of the at least one analyte of interest is a sandwich immunoassay. In some embodiments, the immunoassay used to monitor for the presence of the at least one sample adequacy marker is a sandwich immunoassay. In some embodiments, the immunoassay used to monitor for the presence of the at least one analyte of interest is a competitive immunoassay. In some embodiments, the immunoassay used to monitor for the presence of the at least one sample adequacy marker is a competitive immunoassay. In some embodiments, the immunoassay is a lateral flow assay. In some embodiments, the immunoassay is conducted using a BD Veritor™ System. In some embodiments, the immunoassay is conducted using a BD Veritor™ At-Home COVID-19 Test. In some embodiments, the immunoassay test strip further comprises a negative control line. In some embodiments, the immunoassay test strip further comprises a positive control line. In some embodiments, the positive control line is located on the test strip between the at least one analyte capture line and the at least one sample adequacy line, between the at least one analyte capture line and the negative control line, or between the at least one sample adequacy line and the negative control line. In some embodiments, the positive control line validates the integrity of the immunoassay, proper reagent function, and assures correct testing procedures. In some embodiments, the negative control line validates the integrity of the immunoassay, proper reagent function, and assures correct testing procedures. In some embodiments, the sample is unlabeled upon entry into the flow path. In some embodiments, the sample is labeled upon entry into the flow path. In some embodiments, when the presence of the at least one sample adequacy marker is not detected, the method further comprises displaying an indication that the sample is of insufficient quality. In some embodiments, when the presence of the at least one sample adequacy marker is detected, the method further comprises displaying an indication that the sample is of sufficient quality. In some embodiments, when the presence of the at least one sample adequacy marker is detected in high concentration, the method further comprises displaying an indication that the sample is present in high concentration. In some embodiments, when the presence of the at least one sample adequacy marker is detected in low concentration, the method further comprises displaying an indication that the sample is present in low concentration. In some embodiments, when the presence of the at least one sample adequacy marker is detected and the presence of the at least one analyte of interest is not detected, the method further comprising displaying an indication that the at least one analyte of interest is not detected. In some embodiments, when the presence of the at least one sample adequacy marker is detected and the presence of the at least one analyte of interest is detected, the method further comprises an indication that the analyte of interest is present. In some embodiments, when the presence of the at least one sample adequacy marker is detected and the presence of the at least one analyte of interest is detected in high concentration, the method further comprises displaying an indication that the analyte of interest is present in high concentration. In some embodiments, when the presence of the at least one sample adequacy marker is detected and the presence of the at least one analyte of interest is detected in low concentration, the method further comprises displaying an indication that the analyte of interest is present in low concentration. In some embodiments, when the presence of the at least one sample adequacy marker is detected, the presence of a first analyte of the at least one analyte of interest is detected, and the presence of a second analyte of the at least one analyte of interest is not detected, the method further comprises displaying an indication that the first analyte of the at least one analyte of interest is present and the second analyte of the at least one analyte of interest is not detected. In some embodiments, monitoring for the presence of the at least one sample adequacy marker in the sample comprises visually observing the at least one sample adequacy line for the presence of an optical signal. In some embodiments, monitoring for the presence of the at least one analyte of interest in the sample comprises visually observing the at least one analyte capture line for the presence of an optical signal. In some embodiments, monitoring for the presence of the at least one sample adequacy marker in the sample comprises taking an image of the at least one sample adequacy line. In some embodiments, monitoring for the presence of the at least one analyte of interest in the sample comprises taking an image of the at least one analyte capture line.

Some aspects of the disclosure relate to a test kit. The test kit can include any of the above-described immunoassay test strips; and a computer-readable code identifying a software application configured to analyze an image of the immunoassay test strip to determine a test result based at least in part on the at least one sample adequacy line. In some embodiments, the test result determined by the software application is an invalid test result based at least in part on the software application determining that an optical signal from the at least one sample adequacy line is below a predetermined threshold. In some embodiments, the test result determined by the software application is a valid test result based at least in part on the software application determining that an intensity of an optical signal from the at least one sample adequacy line is above a predetermined threshold.

Embodiments of the present disclosure provided herein are described by way of the following numbered alternatives:

1. An immunoassay test strip comprising:
a flow path configured to receive a sample;
a sample receiving zone coupled to the flow path;
an at least one sample adequacy line; and
an at least one analyte capture line.

2. The immunoassay test strip of Alternative 1, wherein the sample further comprises an at least one sample adequacy marker, the at least one sample adequacy marker in the sample comprises human albumin, and the at least one sample adequacy line includes anti-HSA antibody.

3. The immunoassay test strip of Alternative 1, wherein the sample further comprises an at least one sample adequacy marker, the at least one sample adequacy marker in the sample comprises lactotransferrin, and the at least one sample adequacy line includes anti-lactotransferrin antibody.

4. The immunoassay test strip of any one of Alternatives 2 and 3, wherein the presence and abundancy of the at least one sample adequacy marker in the sample is dependent on the quantity, quality and/or composition of the sample.

5. The immunoassay test strip of any one of Alternatives 1-4, wherein the sample further comprises an at least one analyte of interest.

6. The immunoassay test strip of Alternative 5, wherein the presence and abundancy of the at least one analyte of interest in the sample is dependent on the disease state of the donor.

7. The immunoassay test strip of any one of Alternatives 1-6, wherein the sample is selected from a group consisting of a nasal fluid, mucus, blood, plasma, urine, sweat, or saliva sample collected from a donor.

8. The immunoassay test strip of any one of Alternatives 1-7, wherein the at least one analyte of interest in the sample comprises a marker of infectious disease.

9. The immunoassay test strip of Alternative 8, wherein the at least one analyte of interest in the sample comprises a viral marker.

10. The immunoassay test strip of any one of Alternatives 8-9, wherein the at least one analyte of interest in the sample comprises a marker for SARS-CoV-2.

11. The immunoassay test strip of any one of Alternatives 8-10, wherein the at least one analyte of interest in the sample comprises a marker for influenza.

12. The immunoassay test strip of Alternative 11, wherein the at least one analyte of interest in the sample comprises a marker for influenza A virus.

13. The immunoassay test strip of Alternative 11, wherein the at least one analyte of interest in the sample comprises a marker for influenza B virus.

14. The immunoassay test strip of any one of Alternatives 8-11, wherein the at least one analyte of interest in the sample comprises a marker for influenza and a marker for SARS-CoV-2.

15. The immunoassay test strip of Alternative 8, wherein the at least one analyte of interest in the sample comprises a bacterial marker.

16. The immunoassay test strip of any one of Alternatives 1-12, wherein the donor of the sample is mammalian.

17. The immunoassay test strip of any one of Alternatives 1-13, wherein the donor of the sample is human.

18. The immunoassay test strip of any one of Alternatives 1-17, wherein the test strip is used as part of a direct immunoassay.

19. The immunoassay test strip of any one of Alternatives 1-17, wherein the test strip is used as part of an indirect immunoassay.

20. The immunoassay test strip of any one of Alternatives 1-17, wherein the test strip is used as part of a sandwich immunoassay.

21. The immunoassay test strip of any one of Alternatives 1-17, wherein the test strip is used as part of a competitive immunoassay.

22. The immunoassay test strip of any one of Alternatives 1-21, wherein the test strip is used as part of a lateral flow assay.

23. The immunoassay test strip of any one of Alternatives 1-22, wherein the test strip is used in a BD Veritor™ System.

24. The immunoassay test strip of any one of Alternatives 1-23, wherein the test strip is used in a BD Veritor™ At-Home COVID-19 Test.

25. The immunoassay test strip of any one of Alternatives 1-24, wherein the test strip further comprises a positive control line and/or a negative control line.

26. The immunoassay test strip of Alternative 25, wherein the test strip comprises a positive control line, and wherein the positive control line is located on the test strip between the at least one analyte capture line and the at least one sample adequacy line, between the at least one analyte capture line and a negative control line, or between the at least one sample adequacy line and the negative control line.

27. The immunoassay test strip of Alternative 26, wherein the positive control line validates the integrity of the immunoassay, proper reagent function, and assures correct testing procedures.

28. The immunoassay test strip of Alternative 25, wherein the test strip comprises a negative control line, and wherein the negative control line validates the integrity of the immunoassay, proper reagent function, and assures correct testing procedures.

29. The immunoassay test strip of any one of Alternatives 1-28, wherein the sample is unlabeled upon entry into the flow path.

30. The immunoassay test strip of any one of Alternatives 1-28, wherein the sample is labeled upon entry into the flow path.

31. A method for assessing the adequacy of a sample using an immunoassay test strip, the immunoassay test strip comprising a flow path configured to receive a sample, a sample receiving zone coupled to the flow path, an at least one sample adequacy line, and an at least one analyte capture line, the method comprising:
applying the sample to the immunoassay test strip;
flowing the sample in the flow path to the sample receiving zone;
monitoring for the presence of an at least one analyte of interest in the sample through an immunoassay conducted in the at least one analyte capture line; and
monitoring for the presence of an at least one sample adequacy marker in the sample through an immunoassay conducted in the at least one sample adequacy line; wherein the detection of the at least one sample adequacy marker is used as an indication of the sample quantity, quality and/or composition.

32. The method of Alternative 31, wherein the sample comprises an at least one sample adequacy marker, the at least one sample adequacy marker in the sample comprises human albumin, and the at least one sample adequacy line includes anti-HSA antibody.

33. The method of claim 31, wherein the sample comprises an at least one sample adequacy marker, the at least one sample adequacy marker in the sample comprises lactotransferrin, and the at least one sample adequacy line includes anti-lactotransferrin antibody.

34. The method of any one of Alternatives 31-33, wherein the sample further comprises an at least one analyte of interest.

35. The method of Alternative 34, wherein the presence and abundancy of the at least one analyte of interest in the sample is dependent on the disease state of the donor.

36. The method of any one of Alternatives 31-35, wherein the sample is selected from a group consisting of a nasal fluid, mucus, blood, plasma, urine, sweat, or saliva sample collected from a donor.

37. The method of any one of Alternatives 31-36, wherein the at least one analyte of interest in the sample comprises a marker of infectious disease.

38. The method of Alternative 37, wherein the at least one analyte of interest in the sample comprises a viral marker.

39. The method of any one of Alternatives 37-38, wherein the at least one analyte of interest in the sample comprises a marker for SARS-CoV-2.

40. The method of any one of Alternatives 37-39, wherein the at least one analyte of interest in the sample comprises a marker for influenza.

41. The method of Alternative 40, wherein the at least one analyte of interest in the sample comprises a marker for influenza A virus.

42. The method of Alternative 40, wherein the at least one analyte of interest in the sample comprises a marker for influenza B virus.

43. The method of any one of Alternatives 37-40, wherein the at least one analyte of interest in the sample comprises a marker for influenza and a marker for SARS-CoV-2.

44. The method of Alternative 37, wherein the at least one analyte of interest in the sample comprises a bacterial marker.

45. The method of any one of Alternatives 31-44, wherein the donor of the sample is mammalian.

46. The method of any one of Alternatives 31-45, wherein the donor of the sample is human.

47. The method of any one of Alternatives 31-46, wherein the immunoassay used to monitor for the presence of the at least one analyte of interest is a direct immunoassay.

48. The method of any one of Alternatives 31-47, wherein the immunoassay used to monitor for the presence of the at least one sample adequacy marker is a direct immunoassay.

49. The method of any one of Alternatives 31-46 and 48, wherein the immunoassay used to monitor for the presence of the at least one analyte of interest is an indirect immunoassay.

50. The method of any of Alternatives 31-47 and 49, wherein the immunoassay used to monitor for the presence of the at least one sample adequacy marker is an indirect immunoassay.

51. The method of any one of Alternatives 31-46, 48, and 50, wherein the immunoassay used to monitor for the presence of the at least one analyte of interest is a sandwich immunoassay.

52. The method of any one of Alternatives 31-47, 49, and 51, wherein the immunoassay used to monitor for the presence of the at least one sample adequacy marker is a sandwich immunoassay.

53. The method of any one of Alternatives 31-46, 48, 50, and 52, wherein the immunoassay used to monitor for the presence of the at least one analyte of interest is a competitive immunoassay.

54. The method of any one of Alternatives 31-47, 49, 51, and 53, wherein the immunoassay used to monitor for the presence of the at least one sample adequacy marker is a competitive immunoassay.

55. The method of any one of Alternatives 31-54, wherein the immunoassay is a lateral flow assay.

56. The method of any one of Alternatives 31-54, wherein the immunoassay is conducted using a BD Veritor™ System.

57. The method of any one of Alternatives 31-56, wherein the immunoassay is conducted using a BD Veritor™ At-Home COVID-19 Test.

58. The method of any one of Alternatives 31-57, wherein the immunoassay test strip further comprises a positive control line and/or a negative control line.

59. The method of Alternative 58, wherein the test strip comprises a positive control line, and wherein the positive control line is located on the test strip between the at least one analyte capture line and the at least one sample adequacy line, between the at least one analyte capture line and a negative control line, or between the at least one sample adequacy line and the negative control line.

60. The method of Alternative 59, wherein the positive control line validates the integrity of the immunoassay, proper reagent function, and assures correct testing procedures.

61. The immunoassay test strip of Alternative 58, wherein the test strip comprises a negative control line, and wherein the negative control line validates the integrity of the immunoassay, proper reagent function, and assures correct testing procedures.

62. The method of any one of Alternatives 31-61, wherein the sample is unlabeled upon entry into the flow path.

63. The method of any one of Alternatives 31-61, wherein the sample is labeled upon entry into the flow path.

64. The method of any one of Alternatives 31-63, wherein when the presence of the at least one sample adequacy marker is not detected, the method further comprises displaying an indication that the sample is of insufficient quality.

65. The method of any one of Alternatives 31-63, wherein when the presence of the at least one sample adequacy marker is detected, the method further comprises displaying an indication that the sample is of sufficient quality.

66. The method of Alternative 65, wherein when the presence of the at least one sample adequacy marker is detected in high concentration, the method further comprises displaying an indication that the sample is present in high concentration.

67. The method of Alternative 65, wherein when the presence of the at least one sample adequacy marker is detected in low concentration, the method further comprises displaying an indication that the sample is present in low concentration.

68. The method of any one of Alternatives 65-67, wherein when the presence of the at least one sample adequacy marker is detected and the presence of the at least one analyte of interest is not detected, the method further comprises displaying an indication that the at least one analyte of interest is not detected.

69. The method of any one of Alternatives 65-67, wherein when the presence of the at least one sample adequacy marker is detected and the presence of the at least one analyte of interest is detected, the method further comprises displaying an indication that the analyte of interest is present.

70. The method of Alternative 69, wherein when the presence of the at least one sample adequacy marker is detected and the presence of the at least one analyte of interest is detected in high concentration, the method further comprises displaying an indication that the analyte of interest is present in high concentration.

71. The method of Alternative 69, wherein when the presence of the at least one sample adequacy marker is detected and the presence of the at least one analyte of interest is detected in low concentration, the method further comprises displaying an indication that the analyte of interest is present in low concentration.

72. The method of any one of Alternatives 69-71, wherein when the presence of the at least one sample adequacy marker is detected, the presence a first analyte of the at least one analyte of interest is detected, and the presence of a second analyte of the at least one analyte of interest is not detected, the method further comprises displaying an indication that the first analyte of the at least one analyte of interest is present and the second analyte of the at least one analyte of interest is not detected.

73. The method of any one of Alternatives 31-72, wherein monitoring for the presence of the at least one sample adequacy marker in the sample comprises visually observing the at least one sample adequacy line for the presence of an optical signal.

74. The method of any one of Alternatives 31-73, wherein monitoring for the presence of the at least one analyte of interest in the sample comprises visually observing the at least one analyte capture line for the presence of an optical signal.

75. The method of any one of Alternatives 31-72, wherein monitoring for the presence of the at least one sample adequacy marker in the sample comprises taking an image of the at least one sample adequacy line.

76. The method of any one of Alternatives 31-72, wherein monitoring for the presence of the at least one analyte of interest in the sample comprises taking an image of the at least one analyte capture line.

77. A test kit comprising: the immunoassay test strip of any one of Alternatives 1-30; and a computer-readable code identifying a software application configured to analyze an image of the immunoassay test strip to determine a test result based at least in part on the at least one sample adequacy line.

78. The test kit of Alternative 77, wherein the test result determined by the software application is an invalid test result based at least in part on the software application determining that an intensity of an optical signal from the at least one sample adequacy line is below a predetermined threshold.

79. The test kit of Alternative 77, wherein the test result determined by the software application is a valid test result based at least in part on the software application determining that an intensity of an optical signal from the at least one sample adequacy line is above a predetermined threshold.

### BRIEF DESCRIPTION OF THE DRAWINGS

FIG. 1 depicts an example immunoassay test strip according to the present disclosure, wherein L5 is a Sample Adequacy Line, L4 is a Positive Control line (PC), L3 is a Blank Line, L2 is an Analyte Capture Line for a SARS-Cov-2 marker, L1 is a Negative Control Line (NC), and B2 is a background section.

FIG. 2 depicts a histogram of the distribution of the sample adequacy line intensities in AU units for 369 fluid samples collected and analyzed using an example immunoassay according to the present disclosure.

FIG. 3 depicts a histogram of the distribution of the sample adequacy line intensities in AU for samples collected and analyzed during a clinical trial using an example immunoassay according to the present disclosure.

### DETAILED DESCRIPTION

The development of high-quality rapid diagnostic tests has enabled the deployment of laboratory quality diagnostic assays into environments which historically were unable to process samples and obtain results in real time to enable more relevant patient care. There is however a step in the diagnostic process which has historically remained problematic which is the proper acquisition of the sample to be tested. Poor quality samples will lead to poor quality diagnostic test results. The resulting requirement for highly trained medical professionals to engage in the sample collection step has generally remained an impediment to the extent of deployment of these rapid technologies in all conceivable testing locales.

There has, for this reason, existed a need to enable less highly-trained individuals, including non-laboratory individuals, to collect clinical samples which are valid for analysis. A prior example of such an implementation is the use of Creatinine in urine to normalize the measurement of other biomarkers, such as albumin, to account for the variations in urine output which affects the interpretation of the biomarker of interest.

The value of such an implementation is that the analytical measurements obtained from the clinical sample can be judged for characteristics, such as sample validity. In the testing for abused substances, for example, the individual being tested may be motivated to attempt to alter the analytical outcome via mechanisms such as addition of water to modify the measurement of the diagnostic target. Sample validity tests can detect such alterations and the ensuing results can be judged as valid or invalid as appropriate.

The COVID-19 pandemic has engendered a need for mechanisms to detect viral infection on a scale which is unprecedented. The detection is best accomplished via widespread testing for infection in environments that have not historically been associated with laboratory quality diagnostic testing and, in addition, potentially by individuals not experienced in the collection of valid samples for such testing. Clearly a need has arisen for ensuring the quality of sample collection in widely distributed non-laboratory environments such as airports, long-term care facilities, schools and including the home environment.

Much like the need for sample validity biomarkers in urine, there is a need for identifying and implementing detection of biomarkers in samples derived from the nasal cavity that can be combined with more typical rapid antigen tests for pathogens such as SARS-CoV-2, to ensure quality samples are collected by non-laboratory individuals for evaluation in non-laboratory environments.

Embodiments of the present disclosure relate to systems and techniques for detection of analytes of interest that may be present in biological or non-biological samples such as fluids. Throughout this disclosure, example systems, devices, and methods will be described with reference to collection, testing, and detection of analytes such as those relevant for diagnostic testing related to infectious diseases, but it will be understood that the present technology can be used to collect, test, and detect any particle, molecule, or analyte of interest.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, “a” or “an” may mean one or more than one.

As used herein, the term “about” or “approximately” has its usual meaning as understood by those skilled in the art and thus indicates that a value includes the inherent variation of error for the method being employed to determine a value, or the variation that exists among multiple determinations.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

The terms “function” and “functional” as used herein have their plain and ordinary meaning as understood in light of the specification, and refer to a biological, enzymatic, or therapeutic function.

The present disclosure relates to immunoassay devices, test systems, and methods. As used herein, “analyte” generally refers to a substance to be detected. Analytes of interest may include any detectable substances such as but not limited to antibodies, antigens, antigenic substances, ligands, proteins, haptens, sugars, lipids, nucleic acids, RNA, DNA, amplicons, hormones, small molecules, cytokines, immune cells, immune cell particles, immune cell surfaces, bacterial particles, bacterial surfaces, viral particles, viral surfaces, segments of cells, cell surfaces, and hazardous or non-hazardous drugs or contaminants such as antineoplastic drugs used in the treatment of cancer. Analytes include, but are not limited to, toxins, organic compounds, peptides, microorganisms, amino acids, steroids, vitamins, drugs (including those administered for therapeutic purposes as well as those administered for illicit purposes), drug intermediaries or byproducts, and metabolites of or antibodies to any of the above substances. Specific examples of some analytes include SARS-Cov-2 particles, influenza A particles, influenza B particles, ferritin; creatinine kinase MB (CK-MB); human chorionic gonadotropin (hCG); digoxin; phenytoin; phenobarbitol; carbamazepine; vancomycin; gentamycin; theophylline; valproic acid; quinidine; luteinizing hormone (LH); follicle stimulating hormone (FSH); estradiol, progesterone; C-reactive protein (CRP); lipocalins; IgE antibodies; cytokines; TNF-related apoptosis-inducing ligand (TRAIL); vitamin B2 micro-globulin; interferon gamma-induced protein 10 (IP-10); glycated hemoglobin (Gly Hb); cortisol; digitoxin; N-acetylprocainamide (NAPA); procainamide; antibodies to rubella, such as rubella-IgG and rubella IgM; antibodies to toxoplasmosis, such as toxoplasmosis IgG (Toxo-IgG) and toxoplasmosis IgM (Toxo-IgM); testosterone; salicylates; acetaminophen; hepatitis B virus surface antigen (HBsAg); antibodies to hepatitis B core antigen, such as anti-hepatitis B core antigen IgG and IgM (Anti-HBC); human immune deficiency virus 1 and 2 (HIV 1 and 2); human T-cell leukemia virus 1 and 2 (HTLV); hepatitis B e antigen (HBeAg); antibodies to hepatitis B e antigen (Anti-HBe); influenza virus; thyroid stimulating hormone (TSH); thyroxine (T4); total triiodothyronine (Total T3); free triiodothyronine (Free T3); carcinoembryoic antigen (CEA); lipoproteins, cholesterol, and triglycerides; and alpha fetoprotein (AFP). Drugs of abuse and controlled substances include, but are not intended to be limited to, amphetamine; methamphetamine; barbiturates, such as amobarbital, secobarbital, pentobarbital, phenobarbital, and barbital; benzodiazepines, such as librium and valium; cannabinoids, such as hashish and marijuana; cocaine; fentanyl; LSD; methaqualone; opiates, such as heroin, morphine, codeine, hydromorphone, hydrocodone, methadone, oxycodone, oxymorphone and opium; phencyclidine; and propoxyphene. Additional analytes may be included for purposes of biological or environmental substances of interest. As used herein, an analyte of interest may be present in a sample based on the disease state of the sample's donor.

### Immunoassay

As used herein, the term “immunoassay” has its plain and ordinary meaning as understood in light of the specification, and refers to a biochemical test that measures the presence or concentration of one or more analyte in a solution through the use of an antibody or an antigen. Examples of immunoassays include radioimmunoassays, counting immunoassays, enzyme immunoassays, fluoroimmunoassays, and chemiluminescence immunoassays.

Embodiments of the present disclosure relate to a test strip used in immunoassays that contains a sample adequacy line. Although embodiments of the present disclosure are described herein by reference to the use of said test strips for direct, indirect, sandwich, and competitive immunoassays, it will be understood that the test strip described herein can be used as a component in any analyte detection assay. Further, although embodiments of the present disclosure are described with reference to a test strip, it will be understood that the present disclosure can be implemented in any suitable format, as described in detail below.

As used herein, the term “direct immunoassay” has its usual meaning as understood by those skilled in the art and thus refers to the direct measurement of an analyte of interest by a labeled antibody or labeled antigen specific to that analyte. In some embodiments, the antibody or antigen is fixed onto the test strip and the analyte of interest is contacted with the antibody or antigen in a solution. In some embodiments, the analyte of interest is fixed onto the test strip, and a labeled antibody or antigen is contacted with the analyte in a solution.

As used herein, the term “indirect immunoassay” has its usual meaning as understood by those skilled in the art and thus refers to an assay in which an analyte of interest first binds to an antibody or antigen specific to that analyte and forms a complex, and then a second antibody, analyte, or compound binds to that complex and provides a detectable signal. In some embodiments, the first, or “primary” antibody or antigen is unlabeled. In some embodiments, the analyte of interest is fixed onto the test strip. In some embodiments, the second antibody, analyte, or compound binds to the primary antibody or antigen. In some embodiments, the second antibody, analyte, or compound binds to the analyte. In some embodiments, the second antibody, analyte, or compound binds to the primary antibody or antigen in complex with the analyte.

As used herein, the term “sandwich immunoassay” has its usual meaning as understood by those skilled in the art and thus refers to assay in which an antibody or antigen specific to an analyte of interest is fixed onto the test strip, the analyte of interest then contacts the antibody or antigen in solution and forms a complex, and then a second antibody, analyte, or compound binds to that complex and provides a detectable signal. In some embodiments, the first, or “primary” antibody or antigen is unlabeled. In some embodiments, the second antibody, analyte, or compound binds to the primary antibody or antigen. In some embodiments, the second antibody, analyte, or compound binds to the analyte. In some embodiments, the second antibody, analyte, or compound binds to the primary antibody or antigen in complex with the analyte.

As used herein, the term “competitive immunoassay”, also known as an “inhibition” or “blocking” immunoassay, has its usual meaning as understood by those skilled in the art and thus refers to an assay in which concentration of an analyte of interest in a sample is measured by detecting interference in an expected signal output.

Immunoassays described herein can include a label. Labels can take many different forms, including a molecule or composition bound or capable of being bound to an analyte, analyte analog, detector reagent, or binding partner that is detectable by spectroscopic, photochemical, biochemical, immunochemical, electrical, optical or chemical means. Examples of labels include enzymes, colloidal gold particles (also referred to as gold nanoparticles), colored latex particles, radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulfur particles, protein-adsorbed tellurium particles, protein-adsorbed carbon particles, and protein-coupled dye sacs. The attachment of a compound (e.g., a detector reagent) to a label can be through covalent bonds, adsorption processes, hydrophobic and/or electrostatic bonds, as in chelates and the like, or combinations of these bonds and interactions and/or may involve a linking group.

The term “specific binding partner (or binding partner)” refers to a member of a pair of molecules that interacts by means of specific, noncovalent interactions that depend on the three-dimensional structures of the molecules involved. Typical pairs of specific binding partners include antigen/antibody, hapten/antibody, hormone/receptor, nucleic acid strand/complementary nucleic acid strand, substrate/enzyme, inhibitor/enzyme, carbohydrate/lectin, biotin/(strept)avidin, receptor/ligands, and virus/cellular receptor, or various combinations thereof.

As used herein, the terms “immunoglobulin” or “antibody” refer to proteins that bind a specific antigen. Immunoglobulins include, but are not limited to, polyclonal, monoclonal, chimeric, and humanized antibodies, Fab fragments, F(ab′)2 fragments, and includes immunoglobulins of the following classes: IgG, IgA, IgM, IgD, IbE, and secreted immunoglobulins (slg). Immunoglobulins generally comprise two identical heavy chains and two light chains. However, the terms “antibody” and “immunoglobulin” also encompass single chain antibodies and two chain antibodies.

The immunoassays described herein can be run on a variety of compatible devices, materials, and platforms, for example, but not limited to, base-plates, well-plates, polystyrene microtubes, microtiter plates, lateral flow assays, the BD Veritor™ System, Luminex assays, dry kits, ELISA assays, microarray assays, bead-based assays, fluorescent plate readers, luminescent plate readers, chemical plate readers, optical plate readers, chemiluminescent plate readers, and chip-based assays. Accordingly, although embodiments of the present disclosure are described with reference to an immunoassay test strip, it will be understood that use of a sample adequacy marker to indicate the adequacy of a sample can also be implemented according to the present disclosure in immunoassays that do not use a test strip format, including, but not limited to, an assay performed using a plate-based or well-plate assay, a Luminex assay, an ELISA assay, a bead-based assay, and a microarray assay.

### Lateral Flow Assay

Some aspects of the present disclosure relate to a specialized type of immunoassay, the lateral flow assay. Lateral flow assays are assays that can be performed on lateral flow devices described herein. Lateral flow devices described herein are analytical devices used in lateral flow chromatography. Lateral flow devices may be implemented on a test strip but other forms may be suitable. In the test strip format, a test sample fluid, suspected of containing an analyte, flows (for example by capillary action) through the strip. The strip may be made of bibulous materials such as paper, nitrocellulose, and cellulose. The sample fluid is received at a sample reservoir. The sample fluid can flow along the strip to a capture zone in which the analyte (if present) interacts with a capture agent to indicate a presence, absence, and/or quantity of the analyte. The capture agent can include antibody immobilized in the capture zone.

Signals generated by assays according to the present disclosure are described herein in the context of an optical signal generated by reflectance-type labels (such as but not limited to gold nanoparticle labels). Although embodiments of the present disclosure are described herein by reference to an “optical” signal, it will be understood that assays described herein can use any appropriate material for a label in order to generate a detectable signal, including but not limited to fluorescence-type latex bead labels that generate fluorescence signals and magnetic nanoparticle labels that generate signals indicating a change in magnetic fields associated with the assay.

Implementations of the present disclosure can detect an analyte of interest or sample adequacy marker that occur at various concentrations, including high concentrations. For example, embodiments of the present disclosure can detect analytes using lateral flow devices, test systems, and methods described in International Application No. PCT/US2018/039347, filed Jun. 25, 2018, which is incorporated by reference herein in its entirety. In embodiments implementing lateral flow devices, test systems, and methods described in International Application No. PCT/US2018/039347, filed Jun. 25, 2018, it will be understood that as more of an analyte of interest binds to an analyte capture line, the amount of detectable signal at the analyte capture line decreases, rather than increases.

### Sandwich-Type and Competitive-Type Lateral Flow Assays

Lateral flow assays can be performed in a sandwich or competitive format. Sandwich and competitive format assays described herein will be described in the context of reflective-type labels (such as gold nanoparticle labels) generating an optical signal, but it will be understood that assays may include latex bead labels configured to generate fluorescence signals, magnetic nanoparticle labels configured to generate magnetic signals, or any other label configured to generate a detectable signal. Sandwich-type lateral flow assays include a labeled antibody deposited at a sample reservoir on a solid substrate. After sample is applied to the sample reservoir, the labeled antibody dissolves in the sample, whereupon the antibody recognizes and binds a first epitope on the analyte in the sample, forming a label-antibody-analyte complex. This complex flows along the liquid front from the sample reservoir through the solid substrate to a capture zone (sometimes referred to as a “test line”), where immobilized antibodies (sometimes referred to as “capture agent”) are located. In some cases where the analyte is a multimer or contains multiple identical epitopes on the same monomer, the labeled antibody deposited at the sample reservoir can be the same as the antibody immobilized in the capture zone. The immobilized antibody recognizes and binds an epitope on the analyte, thereby capturing label-antibody-analyte complex at the capture zone. The presence of labeled antibody at the capture zone provides a detectable optical signal at the capture zone. In one non-limiting example, gold nanoparticles are used to label the antibodies because they are relatively inexpensive, stable, and provide easily observable color indications based on the surface plasmon resonance properties of gold nanoparticles. In some cases, this signal provides qualitative information, such as whether or not the analyte is present in the sample. In some cases, this signal provides quantitative information, such as a measurement of the quantity of analyte in the sample.

### Sample Adequacy Marker

In some embodiments the sample is an environmental sample for detecting an analyte in the environment. In some embodiments, the sample is a biological sample from a subject. In some embodiments, the sample is a fluid. In some embodiments, a biological sample can include blood, peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen (including prostatic fluid), Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, nasal fluid, mucus, stool water, pancreatic juice, lavage fluids from sinus cavities, bronchopulmonary aspirates, or other lavage fluids.

As used herein, a “sample adequacy marker” is an analyte, molecule, compound, or substance that can be used to assess the quantity, quality and/or composition of its sample of origin. In traditional immunoassays, a negative result is achieved either when an analyte of interest is not present in the sample, or when the sample is of insufficient adequacy. In the present disclosure, the positive detection of a sample adequacy marker indicates that the sample is present in the immunoassay, and is of high enough concentration and integrity to elicit detection. A sample adequacy marker can be anything that is native to a sample, including constitutive, endogenous, and “housekeeping” molecules. Examples of sample adequacy markers include, but are not limited to, proteins, total proteins, albumin, serum albumin, human albumin, lactotransferrin (lactoferrin), actin, lysozyme, IgA, hemoglobin, mucin, mucin 7, mucin 5ac, mucin 20, trefoil factor 3, cells, cell particles, ammonium, acid phosphates, calcium, the proline-rich protein family, salivary amylase, hormones, Apolipoprotein D, clusterin, prolactin inducible protein, and dermcidin.

### Test Strip for Immunoassay

Immunoassay test strips according to the present disclosure can be used to detect, identify, and in some cases quantify a biologic. A biologic includes chemical or biochemical compounds produced by a living organism which can include a prokaryotic cell line, a eukaryotic cell line, a mammalian cell line, a microbial cell line, an insect cell line, a plant cell line, a mixed cell line, a naturally occurring cell line, or a synthetically engineered cell line. A biologic can include large macromolecules such as proteins, polysaccharides, lipids, and nucleic acids, as well as small molecules such as primary metabolites, secondary metabolites, and natural products.

As disclosed herein, a sample is administered to a test strip through a flow path. In some embodiments, the sample is added to a buffer or solution prior to administration into the flow path. The sample then travels along the flow path into the sample receiving zone. In some embodiments, the test strip comprises a negative control line, which functions to distinguish nonspecific signal from the binding of an analyte of interest to an antibody, analyte, or compound. In some embodiments, the test strip comprises a positive control line, which displays optimal signal for binding. Both positive and negative control lines can validate the integrity of the immunoassay, ensure proper reagent function, and verify correct testing procedures. Some non-limiting implementations of the present disclosure are described in the context of a test strip that includes a test line, a sample adequacy line, a positive control line, and a negative control line, but it will be understood that implementations of the present disclosure need not include a positive control line and/or a negative control line. In some non-limiting embodiments, the sample adequacy line, in addition to indicating whether a sample is adequate or valid, performs the same or similar function as a positive control line.

Certain aspects of the present disclosure include a capture agent. A capture agent includes an immobilized agent that is capable of binding to an analyte, including a free (unlabeled) analyte and/or a labeled analyte. A capture agent includes an unlabeled specific binding partner that is specific for (i) a labeled analyte of interest, (ii) a labeled analyte or an unlabeled analyte, as in a competitive assay, or for (iii) an ancillary specific binding partner, which itself is specific for the analyte, as in an indirect assay. As used herein, an “ancillary specific binding partner” is a specific binding partner that binds to the specific binding partner of an analyte. For example, an ancillary specific binding partner may include an antibody specific for another antibody, for example, goat anti-human antibody. Immunoassay test strips described herein can include a “capture line” that is a region of the lateral flow device where the capture reagent is immobilized. Immunoassay test strips described herein may include more than one capture area, for example, a “primary capture area,” a “secondary capture area,” and so on. In some cases, a different capture reagent will be immobilized in the primary, secondary, and/or other capture areas. Multiple capture areas may have any orientation with respect to each other on the lateral flow substrate; for example, a primary capture area may be distal or proximal to a secondary (or other) capture area along the path of fluid flow and vice versa. Alternatively, a primary capture area and a secondary (or other) capture area may be aligned along an axis perpendicular to the path of fluid flow such that fluid contacts the capture areas at the same time or about the same time.

In some embodiments, capture agents are immobilized such that movement of the capture agent is restricted during normal operation of the immunoassay test strip. For example, movement of an immobilized capture agent is restricted before and after a fluid sample is applied to the immunoassay test strip. Immobilization of capture agents can be accomplished by physical means such as barriers, electrostatic interactions, hydrogen-bonding, bioaffinity, covalent interactions or combinations thereof.

Test strips as described herein also comprise an analyte capture line and a sample adequacy line. The analyte capture line binds to and immobilizes at least one analyte of interest using at least one capture agent. In some embodiments, the binding of an analyte of interest to the analyte capture line produces a detectable signal. Embodiments of the sample adequacy line of the present disclosure are configured to bind to and immobilize at least one sample adequacy marker using at least one capture agent. In some embodiments, the binding of a sample adequacy marker to the sample adequacy line produces a detectable signal. In some embodiments, the signal produced by the analyte capture line is measurably distinct from the sample adequacy line. In some embodiments, at least one signal is fluorescent. In some embodiments, at least one signal is chemiluminescent. In some embodiments, at least one signal is luminescent. In some embodiments, at least one signal is magnetic.

Embodiments of the present disclosure can include lateral flow assays including four distinct lines. For example, a positive control line, a negative control line, an analyte capture line, and a sample adequacy line can be present in any order relative to each other, and arranged any distance with respect to one another, on a test strip. In some embodiments, the lines are distributed such that lines that elicit strong signals are adequately spaced away from lines that elicit weaker signals. It will be understood that a positive control line and/or a negative control line may not be included in a lateral flow assay according to the present disclosure, and that a sample adequacy line according to the present disclosure can be located in any position along the flow path relative to the analyte capture line and any control line that may be present.

As disclosed above, binding of an analyte of interest or sample adequacy marker to a capture line produces a detectable signal or change in signals. This allows for the identification that the analyte of interest and/or the sample adequacy marker is present in the sample. Embodiments of the present disclosure include a sample adequacy line configured to indicate the presence or absence of a sample adequacy marker in a sample. Other embodiments of the present disclosure include a sample adequacy line configured to indicate a quantity, or concentration, of a sample adequacy marker in a sample. In some embodiments, the indication of the presence or absence of a sample adequacy marker in the sample is configured to be read or interpreted visually by a user observing the sample adequacy line. In some embodiments, the presence of the at least one sample adequacy marker is not indicated or detected; in which case the test strip or device detecting signals emitted from the test strip may display an indication that the sample is of insufficient quality. In some embodiments, the presence of the at least one sample adequacy marker is indicated or detected; in which case the test strip or device detecting signals emitted from the test strip may display an indication that the sample is of sufficient quality. In some embodiments, the presence of the at least one sample adequacy marker is indicated or detected in high concentration; in which case the test strip or device detecting signals emitted from the test strip may display an indication that the sample adequacy marker is present in high concentrations. In some embodiments, the presence of the at least one sample adequacy marker is indicated or detected in low concentration; in which case the test strip or device detecting signals emitted from the test strip may display an indication that the sample adequacy marker is present in low concentration. In some embodiments, the presence of the at least one sample adequacy marker is indicated or detected and the presence of the at least one analyte of interest is not indicated or detected; in which case the test strip or device detecting signals emitted from the test strip may display an indication that the at least one analyte of interest is not present, or is not present in a quantity above a limit of detection. In some embodiments, the presence of the at least one sample adequacy marker is indicated or detected, and the presence of the at least one analyte of interest is indicated or detected; in which case the test strip or device detecting signals emitted from the test strip may display an indication that the analyte of interest is present. In some embodiments, the presence of the at least one sample adequacy marker is indicated or detected, and the presence of the at least one analyte of interest is detected in high concentration; in which case the test strip or device detecting signals emitted from the test strip may display an indication that the analyte of interest is present in high concentration. In some embodiments, the presence of the at least one sample adequacy marker is indicated or detected, and the presence of the at least one analyte of interest is detected in low concentration; in which case the test strip or device may display an indication that the analyte of interest is present in low concentration. In some embodiments, the presence of the at least one sample adequacy marker is indicated or detected, the presence of a first analyte of interest of the at least one analyte of interest is indicated or detected, and the presence of a second analyte of interest of the at least one analyte of interest is not indicated or detected; in which case the test strip or device detecting signals emitted from the test strip may display an indication that the first analyte of interest of the at least one analyte of interest is present and the second analyte of interest of the at least one analyte of interest is not present, or is not present in a quantity above a limit of detection.

Test strips and/or cartridges as described herein may be configured for performance of diagnostic and/or non-diagnostic tests. In some embodiments, embodiments of the present disclosure can be implemented in conjunction with systems such as the BD Veritor™ At-Home COVID-19 Test, BD Veritor™ System for Rapid Detection of SARS-CoV-2, the BD Veritor™ System for Rapid Detection of Flu A+B, the BD Veritor™ System for Rapid Detection of Respiratory Syncytial Virus (RSV), the BD Veritor™ System for Rapid Detection of Group A Strep, the BD Veritor™ system, the BD Veritor Plus™ system, and/or components or operations thereof. It will be understood that embodiments of the present disclosure can be implemented in conjunction with any suitable detection system. In some non-limiting examples, test results are read using visual observation of the test strip.

Immunoassay tests strips according to the present disclosure can be included as a component in multiplex assays. Multiplex assays include assays in which multiple, different analytes of interest can be detected, identified, and in some cases quantified. For example, in a multiplex assay device, a primary, secondary, or more capture areas may be present, each specific for one analyte of interest of a plurality of analytes of interest. In one non-limiting example, embodiments of the present disclosure detect a first analyte of interest, including but not limited to, SARS-CoV-2, a second analyte of interest, including but not limited to Influenza A, a third analyte of interest, including but not limited to Influenza B, or any combination of these analytes of interest.

### Methods of Diagnosing a Condition Using Immunoassays According to the Present Disclosure

Some embodiments provided herein relate to methods of using immunoassays to diagnose a medical condition. In some embodiments, the method includes providing an immunoassay as described herein. In some embodiments, the method includes receiving a sample at a sample reservoir of the immunoassay.

In some embodiments, the sample is obtained from a source, including an environmental or biological source. In some embodiments, the sample is suspected of having an analyte of interest. In some embodiments, the sample is not suspected of having an analyte of interest. In some embodiments, a sample is obtained and analyzed for verification of the absence or presence of an analyte. In some embodiments, a sample is obtained and analyzed for the quantity of analyte in the sample. In some embodiments, the quantity of an analyte in a sample is less than a normal value present in healthy subjects, at or around a normal value present in healthy subjects, or above a normal value present in healthy subjects.

In some embodiments, receiving a sample at the sample reservoir of the immunoassay includes contacting a sample with an immunoassay. A sample may contact an immunoassay by introducing a sample to a sample reservoir by external application, as with a dropper or other applicator. In some embodiments, a sample reservoir may be directly immersed in the sample, such as when a test strip is dipped into a container holding a sample. In some embodiments, a sample may be poured, dripped, sprayed, placed, or otherwise contacted with the sample reservoir.

### Example 1

Of the preliminary candidate biomarkers initially selected for evaluation, either one of albumin or lactoferrin were determined to confirm the collection of a viable nasal sample which then ensures the quality of the associated antigen assay. With the introduction of a human biomarker into a rapid antigen test for SARS-CoV-2, the diagnostic test can be reliably deployed to be used by non-laboratory individuals into non-laboratory locales such as at home.

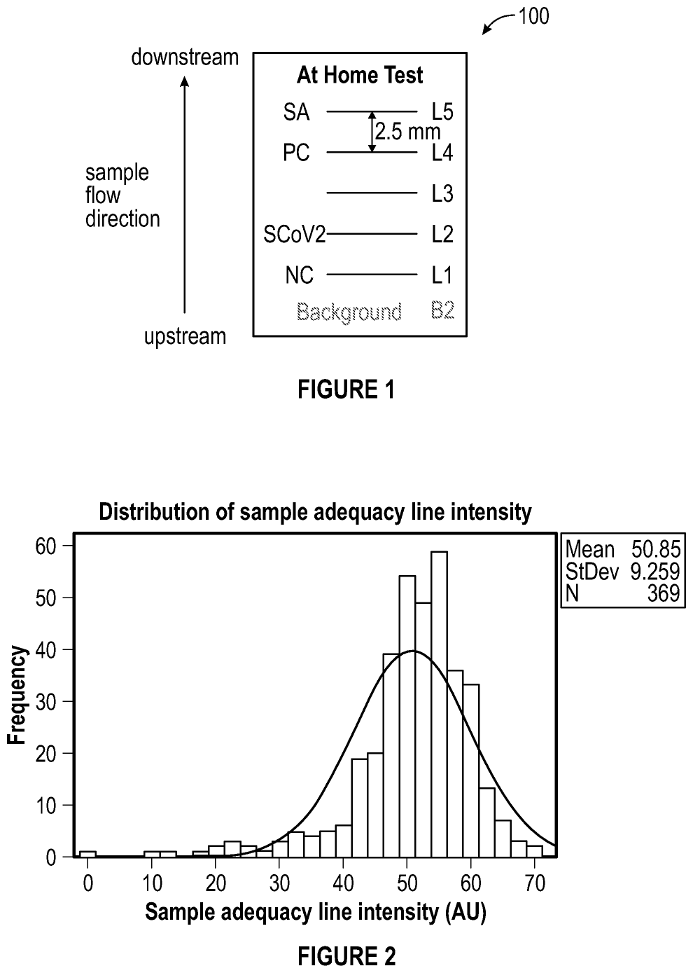

The following example describes preparation and use of a lateral flow assay according to the present disclosure. In this non-limiting example, use of a lateral flow sandwich assay for human serum albumin (HSA) on an At Home COVID-19 test is described. This kind of assay will require the end user (subject under test), in this case, an inexperienced individual, to collect a swab sample from his or her own nasal passage (or the nose of another individual that is the subject under test) without assistance or direction from a trained heath care provider. A portion of a test strip 100 according to this non-limiting example of the lateral flow assay is illustrated in FIG. 1.

To prepare the lateral flow assay, anti-HSA antibody is striped on a sample adequacy line located at the L5 position of a nitrocellulose membrane (solid phase) of the test strip 100, a positive control (PC) line is striped at the L4 position, anti-SARS-CoV-2 antibody is striped on an analyte capture line located at the L2 position, and a negative control (NC) line is striped on the L1 position. The L3 position is left empty in this example, but can be used for other antibodies, such as anti-Flu antibody for a Flu test. The L1 position is upstream and the L5 position is downstream, such that sample flows from background area towards the L5 position. On the upstream side, the nitrocellulose membrane contacts with a conjugate pad (not illustrated) on which there are deposited anti-SARS-CoV-2-gold conjugate, anti-HSA gold conjugate, and a PC line-specific conjugate. The downstream side of the nitrocellulose membrane contacts with an absorbent pad (not illustrated), which acts as sponge to facilitate the sample flowing from the conjugate pad, across the solid phase, in this case the nitrocellulose.

Use of the lateral flow assay will now be described. A user obtains a liquid sample from a nasal swab that was applied to the nasal passage of the subject under test. In one non-limiting example, the liquid sample includes an extraction reagent that has contacted the nasal swab. In one non-limiting example, the extraction reagent contains a buffer in order to maintain a certain pH. The extraction reagent includes a salt solution and a surfactant at this pH to allow specific antibody-antigen binding to occur and to prevent or minimize nonspecific antibody-antigen complexes from forming. The surfactant also assists the sample on the nasal swab to be eluted into the extraction reagent. In another non-limiting example, the liquid sample does not include an extraction reagent. When a liquid sample, or an extracted liquid sample, from a nasal swab is applied to the conjugate pad, the antibody-gold conjugates deposited on the conjugate pad are rehydrated and become solubilized. Specific analyte, such as HSA which is present in the human nose, will bind to the antibody conjugated to the gold particles, forming HSA-antibody-gold complex, and flow towards the reaction zone (in this case, the sample adequacy line located at the L5 position), where another anti-HSA antibody is fixed on the solid phase that can capture the HSA-antibody-gold complex, forming sandwiches and emit signals that can be visually observed by the end user, detected by a detector, and/or imaged for initial or further interpretation or other purposes.

If a swab associated with the sample is not inserted into the nasal passage of the subject under test, or the nasal passage is very dry, the proteins from the nasal passage, such as HSA, are absent or present at a very low concentration. In that event, capture antibody-HSA-antibody-gold sandwich will not form, or will form at a lower than detectable level, and the sample adequacy line located at the L5 position will indicate a negative result, indicating that the sample is inadequate. Another example scenario in which the sample adequacy line located at the L5 position can indicate the sample is inadequate is when a sample is collected from a subject under test that is reluctant or unwilling to allow the user to place a swab in his or her nasal passage (for example, if the subject under test is a child). In this example scenario, the swab may not enter the nasal passage of the subject under test, the swab may not be placed far enough into the nasal passage, or the swab may not be placed into the nasal passage for enough time and/or with sufficient contact. A further example scenario in which the sample adequacy line located at the L5 position can indicate the sample is inadequate is when a sample is collected from a subject under test whose nasal passage is sensitive and/or tender. In this example scenario, the swab may not enter the nasal passage of the subject under test, the swab may not be placed far enough into the nasal passage, or the swab may not be placed into the nasal passage for enough time and/or with sufficient contact. Still another example scenario in which the sample adequacy line located at the L5 position can indicate the sample is inadequate is when a sample is collected using a swab other than a swab indicated for use with the lateral flow assay. For example, the end user may collect the sample using an unapproved or unsuitable swab, resulting in insufficient or inefficient collection of nasal secretions to generate an adequate sample, or the sample cannot be eluted into the extraction reagent due to the material used for the swab tip.

The SARS-CoV-2 assay operates in the same or similar manner as the above-described HSA assay. For example, in the presence of enough viral antigen, capture antibody-SARS-CoV-2 antigen-antibody-detector complex will form on the analyte capture line at the L2 position and emit a detectable signal. In the absence of viral antigen, no sandwich will form and a signal will not be emitted at the analyte capture line, or the signal that is emitted will be below a threshold limit of detection. As long as the liquid phase flows through the PC line, the PC line-specific conjugate will bind to the PC line and a visible line will form, indicating the assay is running appropriately and the assay reagents are viable. In the case that the sample of the subject under test includes an interfering substance, such as human anti-mouse antibody, or a high concentration of rheumatoid factor, the NC line will emit a signal, indicating that the assay should not be reported as positive or that the test result should be invalidated.

### Example 2

Embodiments of the present disclosure can indicate or detect the presence of albumin or lactoferrin at various detection ranges, allowing for a very sensitive sample adequacy test to confirm the collection of a viable nasal sample. In one non-limiting implementation of a lateral flow assay according to the present disclosure, the sample adequacy line indicates the presence of HSA in a sample having a quantity of HSA in the range of about 50 ng/mL to about 1.5 μg/mL Other HSA detection ranges are possible in embodiments of the present disclosure. In another non-limiting implementation of a lateral flow assay according to the present disclosure, the presence of HSA in a sample having less than 50 ng/mL HSA is indicated or detected at the sample adequacy line. In still another non-limiting implementation of a lateral flow assay according to the present disclosure, the lower end of the detection limit for the presence of HSA in a sample is about 100 ng/mL HSA. In still a further non-limiting implementation of a lateral flow assay according to the present disclosure, the presence of HSA in a sample having more than 1.5 μg/mL is indicated or detected at the sample adequacy line. Embodiments of the present disclosure can also indicate or detect lactotransferrin in a sample at various ranges. In one non-limiting implementation of a lateral flow assay according to the present disclosure, the sample adequacy line indicates the presence of lactotransferrin in a sample having about 10 ng/mL lactotransferrin.

### Example 3

Embodiments of the present disclosure can indicate or detect the presence of HSA using a combination of anti-HSA antibodies at a sample adequacy line, allowing for the indication or detection of HSA in a sample at very low concentrations. In one non-limiting implementation of a lateral flow assay according to the present disclosure, two anti-HSA antibodies are applied to the sample adequacy line in a 50/50 ratio. The first anti-HSA antibody is provided by BiosPacific, A15374, and the second anti-HSA antibody is provided by Fitzgerald Industries International, Cat. #10C-CR2116M2, clone 227425. This example implementation resulted in a sample adequacy line that indicated presence of HSA in a sample having HSA at very low concentration. It will be understood that anti-HSA antibody from other providers can be suitably implemented at a sample adequacy line according to the present disclosure. It will also be understood that a ratio different than a 50/50 ratio of two different anti-HSA antibodies, and different combinations of different anti-HSA antibodies, can be suitably implemented at a sample adequacy line according to the present disclosure.

### Example 4

Test results obtained from lateral flow assays according to the present disclosure will now be described. The test results are presented in FIG. 2, which depicts a frequency graph illustrating the distribution of the measured sample adequacy line intensities in AU (arbitrary units) for 369 fluid samples collected and analyzed using an example immunoassay according to the present disclosure. In this experiment, the sample adequacy line of a lateral flow assay according to the present disclosure was striped with anti-HSA antibody. A striping solution containing 1.2 mg/mL BiosPacific anti-HSA antibody A15374 and Fitzgerald anti-HSA antibody 10C-CR2116M2 was deposited on a sample adequacy line of a nitrocellulose membrane at 0.75 μL/cm rate, which corresponds to 0.41 μg of each of the two kinds of antibody being deposited on the test device. The frequency graph of the test results demonstrates that embodiments of the sample adequacy line of the present disclosure can indicate the presence or absence of a sample adequacy marker, in this case HSA, in a sample with sufficient accuracy and reliability to indicate whether the respective sample is adequate for testing.

### Example 5

Clinical trial test results obtained from a lateral flow assay according to the present disclosure will now be described. The present non-limiting example demonstrates the use of an assay including a sample adequacy line according to the present disclosure for determining the presence of SARS-CoV-2 virus, the virus that causes COVID-19, a contagious respiratory illness. Detection of the presence of SARS-CoV-2 in a specimen from a subject can indicate that the subject has contracted the virus causing COVID-19. During the clinical trial held March through May 2021, nasal swab samples were collected by clinical trial participants using the BD Veritor™ At-Home COVID-19 Test (Becton, Dickinson and Company, Franklin Lakes, N.J.), a qualitative test for the detection of SARS-CoV-2 viral proteins in nasal swabs. It will be understood that assays in accordance with the present disclosure may be used for any kind of detection assay that uses a swab to collect a sample.

In this clinical trial, swab samples were collected by trial participants and tested by those same participants at eight point of care sites using a lateral flow assay according to the present disclosure. During non-clinical-trial use of the BD Veritor™ At-Home COVID-19 Test, it will be understood that the individual using the test can collect a nasal swab sample and then test the collected sample at home or in other non-clinical setting, using a lateral flow assay according to the present disclosure that is included in the test. The test can be performed at home with results available in approximately 15 minutes. Step-by-step directions guide the subject through sample collection, sample processing, and instant results interpretation. In the clinical trial, at the point of care sites, the subject performed the BD Veritor™ At-Home COVID-19 Test on themselves, or a parent/legal guardian/companion performed the swabbing, processing of the sample, and interpreting of the test result unassisted as though in a home-setting with only the information available within the associated app on a compatible smartphone, Product Information Leaflet, and Quick Start Guide.

The BD Veritor™ At-Home COVID-19 Test is designed for individuals to test themselves or dependents for proteins from the virus that causes COVID-19. The test is authorized for non-prescription, home use. The test is intended for use by individuals with or without symptoms or other epidemiological reasons to suspect COVID-19 when tested twice over two or three days with at least 24 hours and no more than 48 hours between tests. The test is intended for qualitative detection of SARS-CoV-2 viral proteins in direct anterior nasal swab specimens from individuals ages 2-13 with an adult collecting the specimen and using the test, and age 14 and older with self-sampling. Individuals older than 14 may also have collection by an adult. A positive test result using the BD Veritor™ At-Home COVID-19 Test indicates the tested individual is very likely to have COVID-19 because proteins from the virus that causes COVID-19 were detected in the individual's specimen. A negative test result using the BD Veritor™ At-Home COVID-19 Test indicates that proteins from the virus that causes COVID-19 were not found in the specimen of the tested individual, and the individual is unlikely to have COVID-19. It is possible for the tested individual could have COVID-19 even though the test result is negative.

Eight hundred and ninety-three (893) adult and pediatric human subjects were enrolled in the clinical trial. A swab sample was collected from the nasal passage of each subject using a swab provided in the BD Veritor™ At-Home COVID-19 Test. The sample was collected either directly by the test subject, by the test subject's parent, or by a companion, without assistance from a health care professional (HCP). Each sample was then analyzed using a lateral flow assay by the test subject, by the test subject's parent, or by a companion according to the present disclosure. A reference swab was collected by an HCP and stored in transport medium (universal viral transport medium (UVT) by Becton, Dickinson and Company (Franklin Lakes, N.J.)) and sent to a reference laboratory. The central reference laboratory tested the reference test using the Lyra SARS-CoV-2 Assay (Quidel, San Diego, Calif.).

The lateral flow assay used by test subjects in the clinical trial was implemented on a test strip similar to the test strip 100 described in Example 1 above with reference to FIG. 1. The lateral flow assay test strip was prepared in accordance with Example 3 and Example 4 above. For example, to prepare the lateral flow assay used in the clinical trial, an antibody was striped on a sample adequacy line located at the L5 position of a nitrocellulose membrane (solid phase) of a test strip, similar to the test strip 100, as described in Example 3. The L2 line was striped with a test line antibody, in this case recombinant rabbit monoclonal antibody (HyTest Ltd., Turku, Finland), at 2.4 mg/mL concentration and 0.75 μL/cm striping rate. Each test strip contained 0.814 μg of this antibody. A positive control (PC) line was striped at the L4 position, a negative control (NC) line was striped at the L1 position, and the L3 line was left blank, as described in Example 1. The L1 position was upstream and the L5 position was downstream, such that the sample flowed from the background area towards the L5 position. On the upstream side, the nitrocellulose membrane contacted with a conjugate pad on which there were deposited antibody gold conjugate, and a PC line-specific conjugate. The downstream side of the nitrocellulose membrane contacted with an absorbent pad, which acted as sponge to facilitate the sample flowing from the conjugate pad, across the solid phase, in this case the nitrocellulose.

Each sample collected by a clinical trial participant was applied to a conjugate pad on an individual test strip provided in the BD Veritor™ At-Home COVID-19 Test. The participant interpreted the test result unassisted as though in a home-setting, using the associated app on a compatible smartphone, Product Information Leaflet, and Quick Start Guide. As disclosed in the above examples, samples applied to a test strip that contained a sufficient quantity of viral antigen (for example, at or above the limit of detection for the test) formed an antibody-COVID-19 antigen-antibody-detector complex on the analyte capture line at the L2 position and emitted a signal (in AU units) that was detected using BD Veritor™ At-Home COVID-19 Test application software implemented on a compatible smartphone. A detected signal above a threshold resulted in the sample being identified as containing SARS-CoV-2 virus antigen and the sample test result being reported as “positive” for COVID-19. Samples that did not contain a sufficient quantity of viral antigen did not form such a complex, and a signal (in AU units) was not detected at the analyte capture line. These samples were identified as not containing SARS-CoV-2 virus antigen, and the sample test result was reported as “negative” for COVID-19. The visible line formed by the PC line-specific conjugate bound to the PC line was used as a positive control for each sample, indicating that the assay ran appropriately and that the assay reagents were viable.

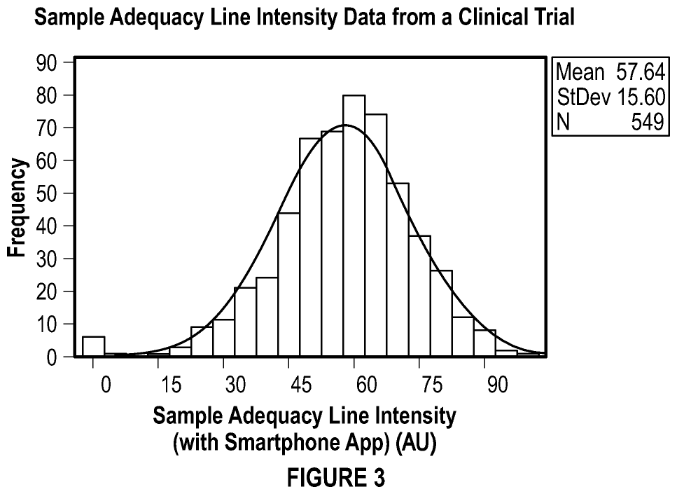

Signals in AU units obtained from lateral flow assays were measured and analyzed using BD Veritor™ At-Home COVID-19 Test application software implemented on a compatible smartphone. The BD Veritor™ At-Home COVID-19 Test overall performance against the Lyra SARS-CoV-2 Assay for symptomatic subjects was 81.3% PPA (positive percent agreement), 99.8% NPA (negative percent agreement), and 98.7% OPA (overall percent agreement). FIG. 3 depicts a histogram of the distribution of the sample adequacy line intensities in AU for samples analyzed during the clinical trial, with frequency on the y-axis and sample adequacy line intensity on the x-axis. The L5 (Sample Adequacy line) values ranged from 0.55 to 98.92 AU, with a mean value of 58.17 AU. As shown in FIG. 3, the intensity measured at the sample adequacy line L5 of some test strips was at or near zero (or the measured intensity was below a predetermined threshold or “cut-off” value for sample adequacy), indicating that HSA was not detected in the sample, even at very low concentrations (or that HSA is present at such a low concentration that it is detected below a predetermined threshold or “cut-off” value for sample adequacy). The absence of HSA (or the presence of HSA at a concentration below the threshold for sample adequacy) in these samples indicated that these samples were inadequate. Example 1 above describes potential, non-limiting reasons that these samples could have been inadequate for testing.

During this clinical trial, a cut-off value of 10.4 AU was used as a threshold or cut-off value to assess whether a particular sample was adequate for testing. For example, an intensity measurement of less than 10.4 AU at the sample adequacy line would cause the respective sample to be deemed inadequate, because the measurement is below the predetermined threshold or cut-off value established for this clinical trial. Such samples were assigned an “invalid” test result by the BD Veritor™ At-Home COVID-19 Test application software. An intensity measurement of more than 10.4 AU at the sample adequacy line would cause the respective sample to be deemed adequate for testing, because the measurement is above the predetermined threshold or cut-off value established for this clinical trial. Such samples were assigned, by the BD Veritor™ At-Home COVID-19 Test application software, a “positive” test result, a “negative” test result, or potentially an “invalid” test result if the test is invalid for reasons unrelated to the intensity measured at the sample adequacy line (for example, based on intensities measured at the positive control line or the negative control line). It will be understood that the threshold or cut-off value for sample adequacy line intensity of 10.4 AU for in this clinical trial is a non-limiting example, and other suitable cut-off values can be implemented in embodiments of the present disclosure.

The results of this clinical trial clearly demonstrated that lateral flow assays including a sample adequacy line according to embodiments of the present disclosure detected SARS-CoV-2 virus antigen in samples with sufficient accuracy and reliability to indicate whether the respective sample was adequate for testing. In tests on most samples, the sample adequacy line displayed strong intensity, indicating that the respective sample was collected properly by the clinical trial participant. In tests on two samples, the sample adequacy line was very weak (0.55 AU and 6.59 AU). These two measured intensities were below the cut-off value of 10.4 AU for sample adequacy established for this clinical trial. As a result, the BD Veritor™ At-Home COVID-19 Test application software judged the test result for these two samples as “invalid.” In a test on a third sample, the measured intensity at the sample adequacy line was 15.09 AU, above the 10.4 AU cut-off value for sample adequacy established for this clinical trial. As a result, the BD Veritor™ At-Home COVID-19 Test application software judged the test result adequate for testing, and in particular, judged the test result to be “negative” based at least in part on the intensity measured at the analyte capture line at position L2 (for this sample, 0.000255 AU). In a test on a fourth sample, the measured intensity at the sample adequacy line was 57.46 AU, above the 10.4 AU cut-off value for sample adequacy established for this clinical trial. As a result, the BD Veritor™ At-Home COVID-19 Test application software judged the test result adequate for testing, and in particular, judged the test result to be “positive” based at least in part on the intensity measured at the analyte capture line at position L2 (for this sample, 119.9 AU). Accordingly, this clinical trial demonstrated that, in two samples, a sample adequacy marker in accordance with the present disclosure was not detected (or was detected at a level below a threshold or cut-off value for sample adequacy), indicating that the quantity, quality and/or composition of the sample of origin was not adequate. In particular, this clinical trial demonstrated that, in two samples, the clinical trial participant did not collect a sample having a quantity, quality and/or composition adequate for testing.

It is to be understood that the description, specific examples and data, while indicating exemplary embodiments, are given by way of illustration and are not intended to limit the various embodiments of the present disclosure. Various changes and modifications within the present disclosure will become apparent to the skilled artisan from the description and data contained herein, and thus are considered part of the various embodiments of this disclosure.

1. An immunoassay test strip comprising:
a flow path configured to receive a sample;
a sample receiving zone coupled to the flow path;
an at least one sample adequacy line; and
an at least one analyte capture line.

2. The immunoassay test strip of claim 1, wherein the sample comprises an at least one sample adequacy marker, the at least one sample adequacy marker in the sample comprises human albumin, and the at least one sample adequacy line includes anti-HSA antibody.

3. The immunoassay test strip of claim 1, wherein the sample comprises an at least one sample adequacy marker, the at least one sample adequacy marker in the sample comprises lactotransferrin, and the at least one sample adequacy line includes anti-lactotransferrin antibody.

4. The immunoassay test strip of claim 1, wherein the sample comprises an at least one sample adequacy marker, and wherein the presence and abundancy of the at least one sample adequacy marker in the sample is dependent on the quantity, quality and/or composition of the sample.

5. (canceled)

6. The immunoassay test strip of claim 4, wherein the sample further comprises an at least one analyte of interest, and wherein the presence and abundancy of the at least one analyte of interest in the sample is dependent on the disease state of the donor.

7. (canceled)

8. The immunoassay test strip of claim 6, wherein the at least one analyte of interest in the sample comprises a marker of infectious disease, a viral marker, a marker for SARS-CoV-2, a marker for influenza, a marker for influenza A virus, or a marker for influenza B virus, wherein an immunoassay conducted in the at least one sample adequacy line is a sandwich-type lateral flow assay, and wherein an immunoassay conducted in the at least one analyte capture line is a sandwich-type lateral flow assay.

9. (canceled)

10. (canceled)

11. (canceled)

12. (canceled)

13. (canceled)

14. The immunoassay test strip of claim 6, wherein the at least one analyte of interest in the sample comprises a marker for influenza and a marker for SARS-CoV-2, wherein an immunoassay conducted in the at least one sample adequacy line is a sandwich-type lateral flow assay, and wherein an immunoassay conducted in the at least one analyte capture line is a sandwich-type lateral flow assay.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. The immunoassay test strip of claim 1, wherein the test strip comprises a positive control line, and wherein the positive control line is located on the test strip between the at least one analyte capture line and the at least one sample adequacy line, between the at least one analyte capture line and a negative control line, or between the at least one sample adequacy line and the negative control line.

27. (canceled)

28. (canceled)

29. (canceled)

30. (canceled)

31. A method for assessing the adequacy of a sample using an immunoassay test strip, the immunoassay test strip comprising a flow path configured to receive a sample, a sample receiving zone coupled to the flow path, an at least one sample adequacy line, and an at least one analyte capture line, the method comprising:
applying the sample to the immunoassay test strip;
flowing the sample in the flow path to the sample receiving zone;
monitoring for the presence of an at least one analyte of interest in the sample through an immunoassay conducted in the at least one analyte capture line; and
monitoring for the presence of an at least one sample adequacy marker in the sample through an immunoassay conducted in the at least one sample adequacy line, wherein the detection of the at least one sample adequacy marker is used as an indication of the sample quantity, quality and/or composition.

32. The method of claim 31, wherein the sample comprises an at least one sample adequacy marker, the at least one sample adequacy marker in the sample comprises human albumin, and the at least one sample adequacy line includes anti-HSA antibody.

33. The method of claim 31, wherein the sample comprises an at least one sample adequacy marker, the at least one sample adequacy marker in the sample comprises lactotransferrin, and the at least one sample adequacy line includes anti-lactotransferrin antibody.

34. (canceled)

35. The method of claim 31, wherein the sample further comprises an at least one analyte of interest, and wherein the presence and abundancy of the at least one analyte of interest in the sample is dependent on the disease state of the donor.

36. The method of claim 31, wherein the sample is a nasal fluid sample collected from a donor.

37. The method of claim 35, wherein the at least one analyte of interest in the sample comprises a marker of infectious disease, a viral marker, a marker for SARS-CoV-2, a marker for influenza, a marker for influenza A virus, or a marker for influenza B virus, wherein an immunoassay conducted in the at least one sample adequacy line is a sandwich-type lateral flow assay, and wherein an immunoassay conducted in the at least one analyte capture line is a sandwich-type lateral flow assay.

38. (canceled)

39. (canceled)

40. (canceled)

41. (canceled)

42. (canceled)

43. The method of claim 35, wherein the at least one analyte of interest in the sample comprises a marker for influenza and a marker for SARS-CoV-2, wherein the immunoassay conducted in the at least one sample adequacy line is a sandwich-type lateral flow assay, and wherein the immunoassay conducted in the at least one analyte capture line is a sandwich-type lateral flow assay.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. The method of claim 31, wherein the test strip comprises a positive control line, and wherein the positive control line is located on the test strip between the at least one analyte capture line and the at least one sample adequacy line, between the at least one analyte capture line and the negative control line, or between the at least one sample adequacy line and the negative control line.

60. (canceled)

61. (canceled)

62. (canceled)

63. (canceled)

64. The method of claim 31, wherein when the presence of the at least one sample adequacy marker is not detected, the method further comprises displaying an indication that the sample is of insufficient quality.

65. The method of claim 31, wherein when the presence of the at least one sample adequacy marker is detected, the method further comprises displaying an indication that the sample is of sufficient quality.

66. The method of claim 65, wherein when the presence of the at least one sample adequacy marker is detected in high concentration, the method further comprises displaying an indication that the sample is present in high concentration.

67. The method of claim 65, wherein when the presence of the at least one sample adequacy marker is detected in low concentration, the method further comprises displaying an indication that the sample is present in low concentration.

68. The method of claim 31, wherein when the presence of the at least one sample adequacy marker is detected and the presence of the at least one analyte of interest is not detected, the method further comprises displaying an indication that the at least one analyte of interest is not detected.

69. The method of claim 31, wherein when the presence of the at least one sample adequacy marker is detected and the presence of the at least one analyte of interest is detected, the method further comprises displaying an indication that the analyte of interest is present.

70. The method of claim 69, wherein when the presence of the at least one sample adequacy marker is detected and the presence of the at least one analyte of interest is detected in high concentration, the method further comprises displaying an indication that the analyte of interest is present in high concentration.

71. The method of claim 69, wherein when the presence of the at least one sample adequacy marker is detected and the presence of the at least one analyte of interest is detected in low concentration, the method further comprises displaying an indication that the analyte of interest is present in low concentration.

72. The method of claim 31, wherein when the presence of the at least one sample adequacy marker is detected, the presence of a first analyte of the at least one analyte of interest is detected, and the presence of a second analyte of the at least one analyte of interest is not detected, the method further comprises displaying an indication that the first analyte of the at least one analyte of interest is present and the second analyte of the at least one analyte of interest is not detected.

73. The method of claim 31, wherein monitoring for the presence of the at least one sample adequacy marker in the sample comprises visually observing the at least one sample adequacy line for the presence of an optical signal.

74. The method of claim 31, wherein monitoring for the presence of the at least one analyte of interest in the sample comprises visually observing the at least one analyte capture line for the presence of an optical signal.

75. The method of claim 31, wherein monitoring for the presence of the at least one sample adequacy marker in the sample comprises taking an image of the at least one sample adequacy line.

76. The method of claim 31, wherein monitoring for the presence of the at least one analyte of interest in the sample comprises taking an image of the at least one analyte capture line.

77. A test kit comprising:
the immunoassay test strip of claim 1; and
a computer-readable code identifying a software application configured to analyze an image of the immunoassay test strip to determine a test result based at least in part on the at least one sample adequacy line.

78. The test kit of claim 77, wherein the test result determined by the software application is an invalid test result based at least in part on the software application determining that an intensity of an optical signal from the at least one sample adequacy line is below a predetermined threshold.

79. The test kit of claim 77, wherein the test result determined by the software application is a valid test result based at least in part on the software application determining that an intensity of an optical signal from the at least one sample adequacy line is above a predetermined threshold.
